(19) United States
(12) Patent Application Publication
(10) Pub. No.: US 2025/0257047 A1
(43) Pub. Date: Aug. 14, 2025

(54) METHOD FOR PREPARING TRIOXANE DERIVATIVES

(71) Applicant: Evonik Oxeno GmbH & Co. KG, Marl (DE)

(72) Inventors: Robert Franke, Marl (DE); Carolin Schneider, Monheim am Rhein (DE); Ralf Jackstell, Rostock (DE); Matthias Beller, Ostseebad Nienhagen (DE)

(73) Assignee: Evonik Oxeno GmbH & Co. KG, Marl (DE)

(21) Appl. No.: 19/046,214

(22) Filed: Feb. 5, 2025

(30) Foreign Application Priority Data
Feb. 14, 2024 (EP) 24157569.5

(51) Int. Cl. C07D 323/06

(52) U.S. Cl. CPC C07D 323/06

(57) ABSTRACT

Method for preparing trioxane derivatives.

The present invention relates to a method for preparing trioxane derivatives.

The object of the present invention was to provide a method for preparing trioxane derivatives. The intention here is to achieve a good yield.

US 2017/0233366 A1 describes a method for preparing trioxane. In this method, an aqueous formaldehyde solution is converted into trioxane at 105° C. in the presence of methanesulfonic acid. Table 1 shows that a formaldehyde conversion of 40% was achieved.

This object is achieved by a method according to claim 1.

Method comprising the method steps of:
a) initially charging an ester of the formula (I):

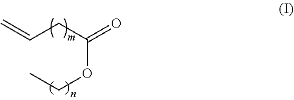

where m is an integer from 1 to 10 and
n is an integer from 0 to 8;
b) adding a compound of the formula (II):

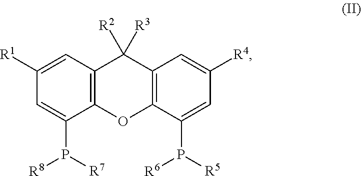

where R¹, R², R³, R⁴, R⁵, R⁶, R⁷, R⁸ are selected from: —H, -(C₁-C₁₂)-alkyl, -(C₆-C₂₀)-aryl;
and, when R¹, R², R³, R⁴, R⁵, R⁶, R⁷, R⁸ are -(C₆-C₂₀)-aryl, the aryl ring may have substituents selected from: -(C₁-C₁₂)-alkyl, -O-(C₁-C₁₂)-alkyl;
c) adding PtI₂;
d) supplying CO and H₂;
e) heating the reaction mixture from a) to d), resulting in conversion of the ester into a compound of the formula (III):

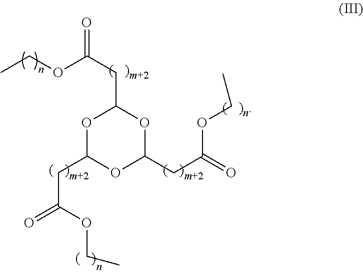

In this method, method steps a) to e) can take place in any desired sequence. However, the addition of CO and H₂ normally takes place after the co-reactants have been initially charged in steps a) to c).

The expression (C₁-C₁₂)-alkyl encompasses straight-chain and branched alkyl groups having 1 to 12 carbon atoms. These are preferably (C₁-C₈)-alkyl groups, more preferably (C₁-C₆)-alkyl, most preferably (C₁-C₄)-alkyl.

The expression (C₆-C₂₀)-aryl encompasses mono-or polycyclic aromatic hydrocarbon radicals having 6 to 20 carbon atoms. These are preferably (C₆-C₁₄)-aryl, more preferably (C₆-C₁₀)-aryl.

In one variant of the method, R², R³, R⁵, R⁶, R⁷, R⁸ are selected from: -(C₁-C₁₂)-alkyl, -(C₆-C₂₀)-aryl.

In one variant of the method, R⁵, R⁶, R⁷, R⁸ are -(C₆-C₂₀)-aryl.

In one variant of the method, R⁵, R⁶, R⁷, R⁸ are —Ph.

In one variant of the method, R² and R³ are -(C₁-C₁₂)-alkyl.

In one variant of the method, R² and R³ are —CH₃.

In one variant of the method, R¹ and R⁴ are —H.

In one variant of the method, the compound (II) has the structure (2):

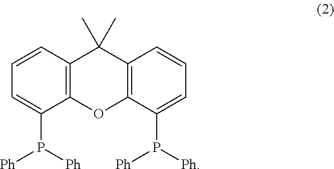 [SMILES: CC1(C)c2cccc(P(c3ccccc3)c3ccccc3)c2Oc2c(P(c3ccccc3)c3ccccc3)cccc21]

In one variant of the method, m is an integer from 5 to 9.

In one variant of the method, m is 7.

In one variant of the method, n is an integer from 0 to 4.

In one variant of the method, n is 0.

In one variant of the method, the ester has the structure (1):

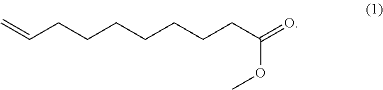

In one variant of the method, CO and H₂ are supplied at a pressure within a range from 1 MPa (10 bar) to 6 MPa (60 bar).

In one variant of the method, CO and H₂ are supplied at a pressure within a range from 3 MPa (30 bar) to 5 MPa (50 bar).

In one variant of the method, the reaction mixture is heated to a temperature in the range from 40° C. to 100° C.

In one variant of the method, the reaction mixture is heated to a temperature in the range from 50° C. to 80° C.

In one variant of the method, said method includes the additional method step d′) of: d′) adding a solvent.

In one variant of the method, the solvent is selected from: THF, MTBE, DCM, ACN, heptane, DMF, toluene, xylene, mesitylene, dibenzyltoluene.

In one variant of the method, the solvent is toluene.

In addition to the method, a compound is also claimed.

Compound of the formula (IV):

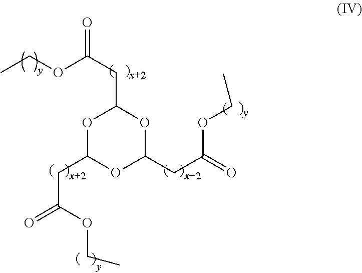

where x is an integer from 1 to 10 and
y is an integer from 0 to 8.

In one embodiment, x is an integer from 5 to 9.

In one embodiment, x is 7.

In one embodiment, y is an integer from 0 to 4.

In one embodiment, y is 0.

In one embodiment, the compound of the formula (IV) has the structure (4):

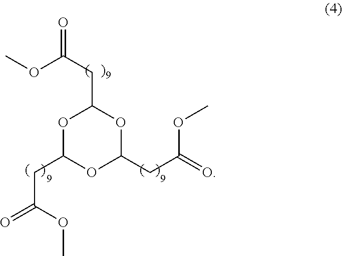

The invention will be elucidated in more detail hereinbelow with reference to an exemplary embodiment.

### Synthesis of trimethyl 10,10′, 10″-(1,3,5-trioxane-2,4,6-triyl)tris(decanoate) (4)

150 ml (132 g) of methyl dec-9-enoate (1), 100 ml of absolute toluene, 320 mg of PtI₂ (0.1 mol % relative to (1)), 620 mg of Xantphos (2) (0.15 mol % relative to (1)) are placed under argon in a 450 ml high-pressure autoclave (Parr Instruments) equipped with stirring and electronic pressure transducer. The autoclave is pressurized to 40 bar with synthesis gas (H₂:CO=1:1) and the reaction is carried out at 60° C. with stirring (>500 min⁻¹). The reaction time is 10 h. The gas consumption is readjusted so that the reaction takes place at approx. 40 bar. After 10 h, the reaction is stopped, the autoclave is cooled down, the gas is vented and the autoclave is flushed 4 times with 30 bar of nitrogen. The reaction solution is transferred to a 500 ml Schlenk vessel.

A GC is run. The GC yield of methyl 11-oxoundecanoate (3) is: 98%, (n:iso selectivity=98.2:1.8).

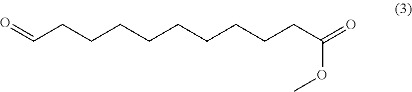

The mixture is then distilled under fine vacuum at 10⁻³ torr (K_{P}=100° C.). A colourless liquid is obtained (146 g =96%).

This liquid crystallizes completely within 24 h to a solid identified by ¹H-NMR, ¹³C-NMR and MS analysis as trimethyl 10,10′,10″-(1,3,5-trioxane-2,4,6-triyl)tris(decanoate) (4). The purity is >99%.

NMR (CDCl₃, 300 MHz):

¹H: 4.75 t (3H, J_{HH}=5.3 Hz), 3.59 s (9H), 2.23 (6H, J_{HH}=7.5 Hz), 1.6-1.5 m (12H) 1.38-1.16 m (36H) ¹³C: 174.28 s, 101.65 s, 51.40 s, 34.39 s, 34.08 s, 29.38 s, 29.32 s, 29.20 s, 29.11 s, 24.93 s, 23.53 s

MS (70 ev, MZ (%)): 186(18), 171(44), 143(27), 139(65), 129(9), 121(17), 111(18), 98(36), 97(31), 8(78), 74(100), 69(43), 59(29), 57(17), 55(64).

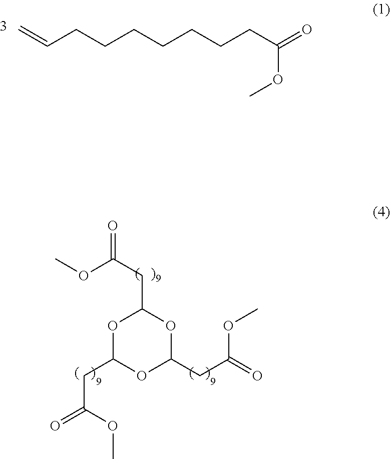

### Reaction Conditions:

Ester (1), 0.1 mol % PtI₂, 0.15 mol % Xantphos (2), solvent:toluene, p(CO/H₂): 40 bar, T: 60° C., t: 10 h.

As shown by the experimental results, the object is achieved by the method according to the invention.

1. Method comprising the method steps of:
a) initially charging an ester of the formula (I): 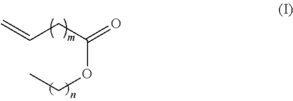
where m is an integer from 1 to 10 and
n is an integer from 0 to 8;
b) adding a compound of the formula (II): 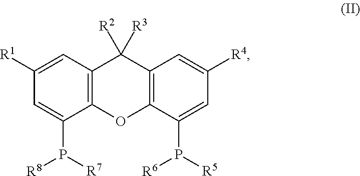
where R¹, R², R³, R⁴, R⁵, R⁶, R⁷, R⁸ are selected from: —H, -(C₁-C₁₂)-alkyl, -(C₆-C₂₀)-aryl;
and, when R¹, R², R³, R⁴, R⁵, R⁶, R⁷, R⁸ are -(C₆-C₂₀)-aryl, the aryl ring may have substituents selected from: -(C₁-C₁₂)-alkyl, -O-(C₁-C₁₂)-alkyl;
c) adding PtI_{2;}
d) supplying CO and H₂;
e) heating the reaction mixture from a) to d), resulting in conversion of the ester into a compound of the formula (III):

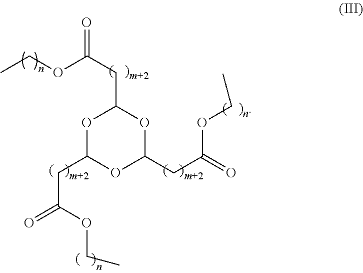

2. Method according to claim 1,
where R¹, R², R³, R⁴, R⁵, R⁶, R⁷, R⁸ are selected from: -(C₁-C₁₂)-alkyl, -(C₆-C₂₀)-aryl.

3. Method according to claim 1,
where R⁵, R⁶, R⁷, R⁸ are -(C₆-C₂₀)-aryl.

4. Method according to claim 1,
where R² and R³ are -(C₁-C₁₂)-alkyl.

5. Method according to claim 1,
where R¹ and R⁴ are —H.

6. Method according to claim 1,
where the compound (II) has the structure (2):

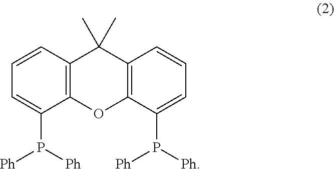 [SMILES: CC1(C)c2cccc(P(c3ccccc3)c3ccccc3)c2Oc2c(P(c3ccccc3)c3ccccc3)cccc21]

7. Method according to claim 1,
where m is an integer from 5 to 9.

8. Method according to claim 1,
where n is an integer from 0 to 4.

9. Method according to claim 1,
where the ester has the structure (1):

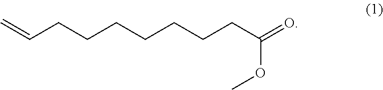

10. Method according to claim 1,
where CO and H₂ are supplied at a pressure within a range from 1 MPa (10 bar) to 6 MPa (60 bar).

11. Method according to claim 1,
where the method includes an additional method step d′) of:
d′) adding a solvent.

12. Compound of the formula (IV): 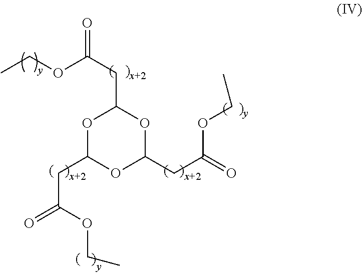
where x is an integer from 1 to 10 and
y is an integer from 0 to 8.

13. Compound according to claim 12,
where x is an integer from 5 to 9.

14. Compound according to claim 12,
where y is an integer from 0 to 4.

15. Compound according to claim 12,
where the compound of the formula (IV) has the structure (4): [chemical structure]
